(19) United States
(12) Patent Application Publication
(10) Pub. No.: US 2023/0119053 A1
(43) Pub. Date: Apr. 20, 2023

(54) Process for Preparing Sartan Active Compounds Having a Tetrazole Ring

(71) Applicant: SANOFI, Paris (FR)

(72) Inventors: Paul CRUCIANI, Paris (FR); Isabelle GALIBOURG, Paris (FR); Bernard GRIMAUD, Paris (FR)

(73) Assignee: SANOFI, Paris (FR)

(21) Appl. No.: 17/937,405

(22) Filed: Sep. 30, 2022

(30) Foreign Application Priority Data
Oct. 1, 2021 (EP) 21306383.7

(51) Int. Cl. C07D 257/06; C07D 403/10

(52) U.S. Cl. CPC C07D 257/06; C07D 403/10

(57) ABSTRACT

The present disclosure relates to a process for manufacturing at least one sartan active compound of formula (I)

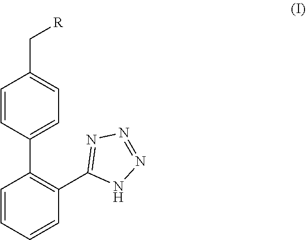

wherein R is selected from a group of formulas (1), (2), (3), (4) and (5), comprising the tetrazolylation of one compound of formula (II)

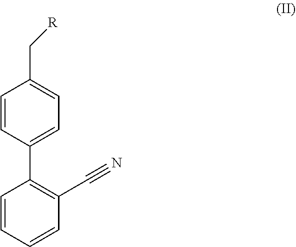

wherein R is as previously defined, in a reactional medium with at least one azide derivative, wherein benzylic azide impurities formed during said tetrazolylation are converted into aldehyde derivatives.

### CROSS-REFERENCE TO RELATED APPLICATION

This application claims priority to European Patent Application No. 21306383.7, filed Oct. 1, 2021, the entire contents of which are incorporated herein by reference.

### FIELD OF THE INVENTION

The invention belongs to the field of pharmacy, and particularly relates to a method for synthesizing a high-purity sartan active compounds having a tetrazole ring.

### BACKGROUND OF THE INVENTION

Irbesartan, losartan, valsartan and candesartan are all prescription angiotensin receptor blocker (ARB) drugs, which are also known as “sartans” active compounds. “Sartans” are a class of drugs used to treat patients with high blood pressure to help prevent heart attacks and stroke. In particular, irbesartan is an antihypertensive drug that is an angiotensin II type I (AII1)-receptor antagonist for the treatment of hypertension. The drug is also the first major antihypertensive drug approved for the treatment of patients with hypertension, type 2 diabetes, and kidney disease.

Various methods of preparing irbesartan and related compounds were disclosed in the literature. One of the methods implements a tetrazolylation step as typically described in EP 0 708 103 for irbesartan.

As far as Irbesartan is concerned, said tetrazolylation step may be performed from the following intermediate compound: 2-n-butyl-1-[(2′-cyanobiphenyl-4-yl)methyl]-4-spirocyclopentane-2-imidazoline-5-one or 2-n-butyl-3-[(2′-cyanobiphenyl-4-yl)methyl]-1,3-diazaspiro[4.4]non-1-ene-4-one, which is tetrazolylated with an alkali metal azide (also named alkaline azide or alkali azide in the present text) and a base to form irbesartan. However, said route of synthesis presents a major drawback consisting in forming azido impurities like (5-(4′-(azidomethyl)-[1,1′-biphenyl]-2yl)-1H-nitrile (also named 4′-(azidomethyl)-[1,1′-biphenyl]-2-carbonitrile or azido nitrile in the present text) and (5-(4′-(azidomethyl)-[1,1′-biphenyl]-2yl)-1H-tetrazole (also named 5-(4′-(azidomethyl)-[1,1′-biphenyl]-2yl)-1H-1,2,3,4-tetrazole or azido tetrazole in the present text) during this tetrazolylation.

The azido impurity (5-(4′-(azidomethyl)-[1,1′-biphenyl]-2yl)-1H-tetrazole, has recently been discovered as having mutagenic properties. A mutagen is a chemical substance that can cause a change in the DNA of a cell. These mutations may increase the risk of cancer but the specific risk for this azido impurity to cause cancer in humans is unknown. Accordingly, health authorities now request to ensure that the level of azido impurity stay below the toxicological threshold of concern (TTC). As azido impurities must no longer be detectable in the finished medicinal product, improvements in the existing manufacturing processes for producing sartans having a tetrazole ring, thus implementing a tetrazolylation step, have been required.

The present invention achieves this need by providing a manufacturing process allowing to degrade the azido impurities, more particularly the benzylic azides impurities. The present invention more particularly provides an impurity profile of the obtained sartans, compliant with these required low level of benzylic azides impurities which may typically be less than or equal to 10 ppm, and even less than or equal to 5 ppm with respect to the total amount of the sartan compound. Accordingly, said sartans having a tetrazole ring may be manufactured with high quality in the framework of the present invention.

### SUMMARY OF THE INVENTION

The present invention, starting from the usually used cyano derivative intermediates suitable for the tetrazolylation, provides an advantageous process useful for preparing sartan active compounds having a tetrazole ring, in particular irbesartan, having significant lower amounts in azido impurities like (5-(4′-(azidomethyl)-[1,1′-biphenyl]-2yl)-1H-nitrile and (5-(4′-(azidomethyl)-[1,1′-biphenyl]-2yl)-1H-tetrazole.

Herein is described a process for manufacturing at least one sartan active compound of formula (I)

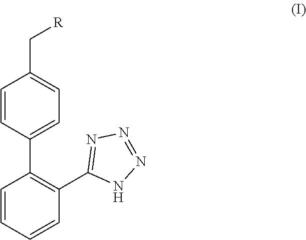

wherein R is selected from a group of formulas (1), (2), (3), (4) and (5):

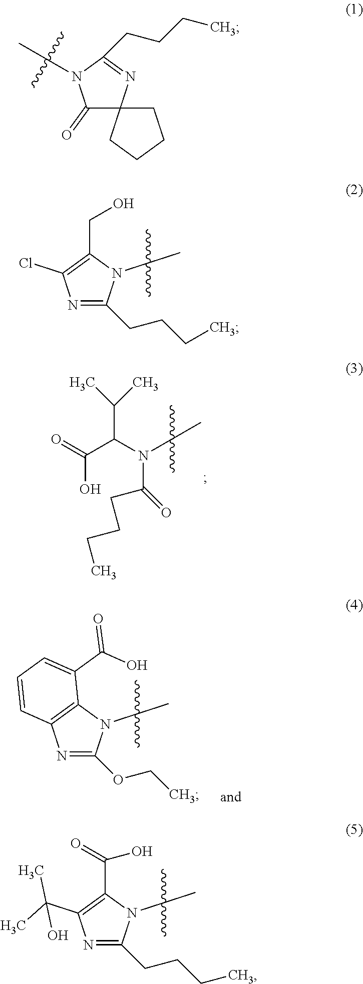 [SMILES: *N1C(=O)C2(CCCC2)N=C1CCCC ; *n1c(CCCC)nc(Cl)c1CO ; *N(C(=O)CCCC)C(C(=O)O)C(C)C ; *n1c(OCC)nc2cccc(C(=O)O)c21 ; *n1c(CCCC)nc(C(C)(C)O)c1C(=O)O]

[symbol]

being the attachment site,
comprising the tetrazolylation of one compound of formula (II)

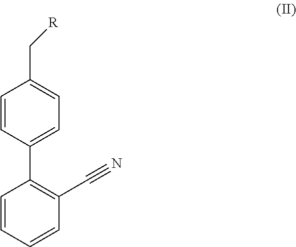

wherein R is as previously defined,
in a reactional medium with at least one azide derivative, wherein benzylic azide impurities formed during said tetrazolylation are converted into aldehyde derivatives.

The present manufacturing process further presents the advantage of implementing the classical N−1 intermediate that is usually used for the tetrazolylation, i.e. the intermediate having a cyanophenyl moiety, and in particular a compound of formula (II) as defined herein after. It means that the required process adjustments are quite light with respect to the existing process while providing the required very high level of purity of the sartan compound.

### DETAILED DESCRIPTION OF THE INVENTION

The disclosure relates to a process for manufacturing at least one sartan active compound of formula (I)

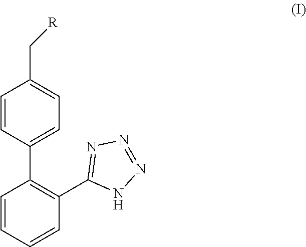

wherein R is selected from a group of formulas (1), (2), (3), (4) and (5):

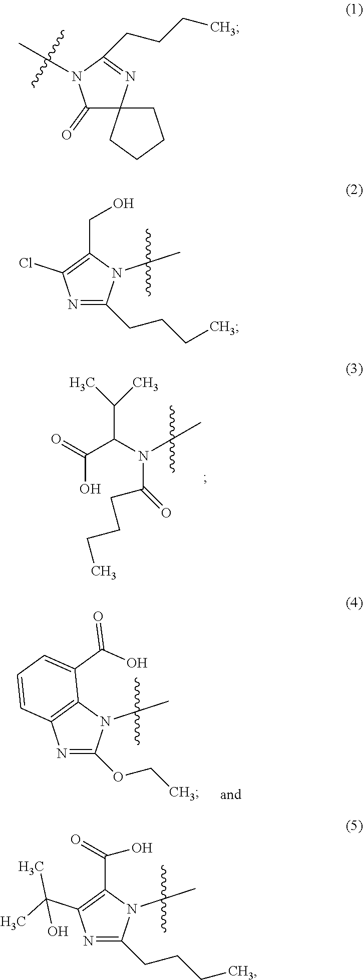

[symbol]

being the attachment site,
comprising the tetrazolylation of one compound of formula (II)

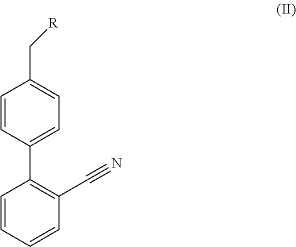

wherein R is as previously defined,
in a reactional medium with at least one azide derivative, wherein benzylic azide impurities formed during said tetrazolylation are converted into aldehyde derivatives.

As used herein, the term “ambient temperature” or “room temperature” (also named RT) refers to a temperature ranging from 15° C. to 35° C., more particularly from 25° C. to 35° C.

As used herein “inert atmosphere” means an atmosphere not suitable for an oxidation. This means that the atmosphere is free of oxygen. For instance, an inert atmosphere may be nitrogen gas or argon gas.

In the sense of the present disclosure, “benzylic azide impurities” cover all the by-products or impurities likely to be produced by nucleophilic substitution with alkali metal azides of compounds which comprise in their structure at least one activated carbon atom. “Activated carbon atom” means in the context of the present disclosure, a carbon atom which bears a leaving group such as a halogen atom (chlorine, bromine or iodine atom), an alcohol group, a tosylate group, a mesylate group, an alkylphosphate group, an ester group, or an amide group and so on. Such “activated carbon atom” is more particularly a carbon atom linked to a phenyl ring thus forming an activated benzylic structure. Said “benzylic azide impurities” can be thus present during a tetrazolylation step starting from an intermediate compound having a cyanophenyl moiety and implementing an alkali metal azide.

Herein is further provided a process according to the present disclosure, wherein said benzylic azide impurities are converted into aldehyde derivatives by performing an oxidation step followed by a hydrolysis step.

The process in accordance with the present disclosure is more detailed herein after.

### Conversion of the Benzylic Azide Impurities: Oxidation Followed by Hydrolysis

As mentioned above, in the conventional processes involving cyano derivative intermediates for the tetrazolylation, two well-known mutagenic benzylic azide impurities are formed, namely azido nitrile of formula (A) and azido tetrazole of formula (B) as shown below:

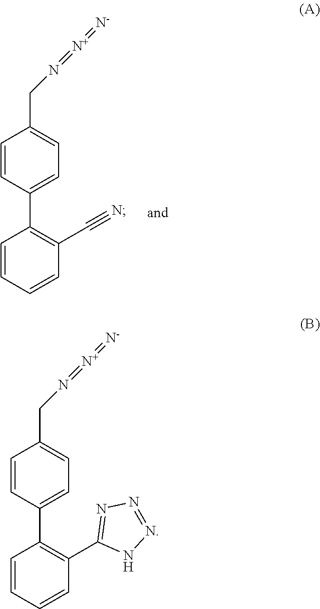 [SMILES: N#Cc1ccccc1-c1ccc(CN=[N+]=[N-])cc1 ; [N-]=[N+]=NCc1ccc(-c2ccccc2-c2nnn[nH]2)cc1]

Thus, herein is further provided a process according to the present disclosure, wherein said benzylic azide impurities comprise at least the compounds of formula (A) and (B) as defined above.

Actually, these two benzylic azide impurities may be formed during the tetrazolylation of a compound of formula (II) as defined in the present disclosure from several different potential precursors.

A process in accordance with the present disclosure allows the degradation of these benzylic azide impurities, and more particularly their conversion into corresponding aldehyde derivatives which are assessed as class 5 impurities, that is to say they are considered as non-mutagenic compounds.

More particularly, this conversion consists in an oxidation step followed by a hydrolysis step. The oxidation step starting from benzylic azide impurities allows to obtain in situ the corresponding chemically unstable benzylic imines.

Then, these unstable benzylic imines, when put in contact with water, are transformed into the corresponding aldehyde derivatives via hydrolysis.

More particularly, the above-mentioned aldehyde derivatives may be those of formulas (A1) (also named nitrile aldehydic impurity in the present disclosure) and (B1) (also named tetrazole aldehydic impurity in the present disclosure), which derive from benzylic azide impurities respectively of formulas (A) and (B):

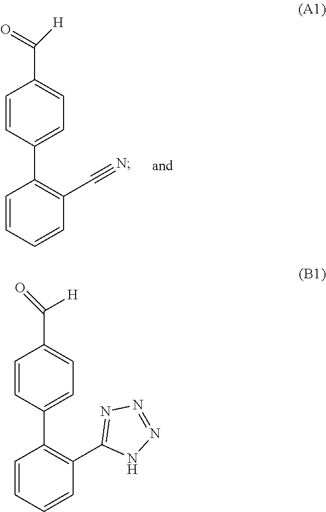

Herein is further provided a process according to the present disclosure, wherein the so-obtained aldehyde derivatives comprise at least the compound of formula (B1)

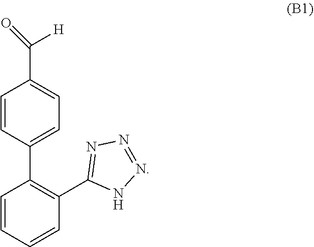 [SMILES: O=Cc1ccc(-c2ccccc2-c2nnn[nH]2)cc1]

Herein is further provided a process according to the present disclosure, wherein the obtained sartan active compound of formula (I) as defined in the present disclosure contains less than 10 ppm, in particular less than 5 ppm, and more particularly less than 1 ppm of a compound of formula (B1)

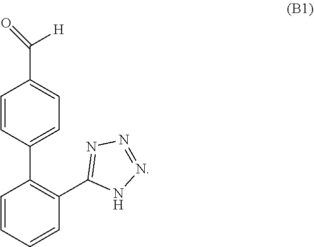 [SMILES: O=Cc1ccc(-c2ccccc2-c2nnn[nH]2)cc1]

The conversion in a process in accordance with the present disclosure may be performed by contacting said benzylic azide impurities with at least ferrous ions (Fe²⁺).

Thus, herein is further provided a process according to the present disclosure, wherein said benzylic azide impurities are converted into aldehyde derivatives by contacting said benzylic azides impurities with at least ferrous ions.

The supply of said ferrous ions can be carried out according to two variants.

According to a first variant, said ferrous ions may be formed in situ by reduction of ferric ions (Fe³⁺), in particular in the presence of a polar aprotic solvent having reductive properties which is defined herein after.

Thus, herein is further provided a process according to the present disclosure, wherein said ferrous ions are formed in situ by reduction of ferric ions, in particular in the presence of a polar aprotic solvent having reductive properties.

Among the compounds which can generate the ferric ions can be cited for instance FeCl₃, FePO₄, FeI₃, FeF₃, FeBr₃, Fe₂(SO₄)₃, Fe₂(C₂O₄)₃, Fe(OH)₃, FeCl₃.6H₂O, FeF₃.3H₂O, Fe₄(P₂O₇)₃, Fe₄(Fe(CN)₆)₃, or Fe(H₂PO₂)₃, in particular FeCl₃, FePO₄, FeI₃, FeF₃, FeBr₃, Fe₂(SO₄)₃, Fe₂(C₂O₄)₃, or Fe(OH)₃, such as FeCl₃. Advantageously, the compound which can generate said ferric ions is anhydrous, and is in particular anhydrous FeCl₃.

Thus, herein is further provided a process according to the present disclosure, wherein said ferric ions are generated from FeCl₃, FePO₄, FeI₃, FeF₃, FeBr₃, Fe₂(SO₄)₃, Fe₂(C₂O₄)₃, Fe(OH)₃, FeCl₃.6H₂O, FeF₃.3H₂O, Fe₄(P₂O₇)₃, Fe₄(Fe(CN)₆)₃, or Fe(H₂PO₂)₃, in particular from FeCl₃, FePO₄, FeI₃, FeF₃, FeBr₃, Fe₂(SO₄)₃, Fe₂(C₂O₄)₃, or Fe(OH)₃, for instance FeCl₃.

During this conversion, the used temperature may be of 80° C. to 150° C., in particular of 100° C. to 135° C., and under inert atmosphere when ferric ions such as FeCl₃, FePO₄, FeI₃, FeF₃, FeBr₃, Fe₂(SO₄)₃, Fe₂(C₂O₄)₃, Fe(OH)₃, FeCl₃.6H₂O, FeF₃.3H₂O, Fe₄(P₂O₇)₃, Fe₄(Fe(CN)₆)₃, or Fe(H₂PO₂)₃, in particular from FeCl₃, FePO₄, FeI₃, FeF₃, FeBr₃, Fe₂(SO₄)₃, Fe₂(C₂O₄)₃, or Fe(OH)₃, for instance FeCl₃, are used.

According to a second variant, said ferrous ions may be incorporated directly in the reaction medium.

Among the compounds which can generate said ferrous ions can be cited for instance FeCl₂, FeBr₂, FeI₂, FeF₂, FeCO₃, FeSO₄, Fe₃(PO₄)₂, Fe₂SiO₄, Fe(OH)₂, Fe(C₂H₃O₂)₂, FeSO₄.H₂O, FeSO₄.7H₂O, FeSO₄.4H₂O, FeS, FeI₂.4H₂O, FeF₂.4H₂O, FeCl₂.4H₂O, FeCl₂.2H₂O, FeBr₂.6H₂O, Fe(NO₃)₂.6H₂O, Fe(NO₃)₂ or Fe(AlO₂)₂, in particular FeCl₂, FeBr₂, FeI₂, FeF₂, FeCO₃, FeSO₄, Fe₃(PO₄)₂, Fe₂SiO₄, Fe(OH)₂, or Fe(C₂H₃O₂)₂, such as FeCl₂.

Besides, the conversion of the benzylic azide impurities can be performed to two embodiments, either simultaneously or subsequently to said tetrazolylation.

According to one embodiment, the conversion may be performed simultaneously to said tetrazolylation.

According to said embodiment, the total amount of ferrous ions present in the reaction medium may be controlled. More particularly, the total amount of ferrous ions may be introduced in a catalytic amount. Said catalytic amount allows to reduce, avoid or prevent the thermic instability of the azide derivative.

More particularly, the ferrous ions may be present at a catalytic amount in the basic medium containing the azide derivatives, in particular in a molar percentage ranging from 0.005% to 0.1%, in particular from 0.01% to 0.05% with respect to the amount of the compound of formula (II) as defined in the present disclosure.

Still according to said embodiment, when the sartan active compound of formula (I) is irbesartan, the ferrous ions may be present in a molar percentage ranging from 0.005% to 0.1%, in particular from 0.01% to 0.05% with respect to the amount of 2-n-butyl-3-[(2′-cyanobiphenyl-4-yl)methyl]-1,3-diazaspiro[4.4]non-1-en-4-one.

According to another embodiment, the conversion may be performed subsequently to said tetrazolylation.

According to said embodiment, the total amount of ferrous ions present in the reaction medium may be less critical as in the first embodiment, the tetrazolylation step being completed.

Thus, the ferrous ions may be present in the reaction medium in any amount, for instance in a catalytic amount or in a stoichiometric amount.

For instance, the ferrous ions may be present in a molar percentage ranging from 0.005% to 10%.

### Tetrazolylation

Tetrazolylation means a conversion of nitriles into tetrazoles.

Tetrazolylation by reaction with azide derivative, for instance tributyltin azide or alkali metal azide such as sodium azide, and a base such as triethylamine hydrochloride is described in the literature.

Thus, for instance, the preparation of 2-n-butyl-3-[[2′-(tetrazol-5-yl)biphenyl-4-yl]methyl]-1,3-diazaspiro[4.4]non-1-en-4-one (also named irbesartan) from 2-n-butyl-3-[(2′-cyanobiphenyl-4-yl)methyl]-1,3-diazaspiro[4.4]non-1-en-4-one (also named Spiro Methyl Biphenyl Nitrile in the present disclosure), by heating at reflux in the presence of azide of tributyltin is known.

EP 0 708 103 showed that the use of 1-methylpyrrolidin-2-one as a solvent at a temperature of about 150° C., namely at a temperature at which reflux is observed, is particularly advantageous for overcoming the drawback.

Accordingly, a privileged route for performing this tetrazolylation is by reaction of compound of formula (II) as defined in the present disclosure with an azide derivative and one base in an inert polar aprotic solvent at a temperature below the reflux temperature and under inert atmosphere.

The following scheme 1 illustrates succinctly the tetrazolylation from a compound of formula (II) to obtain a compound of formula (I).

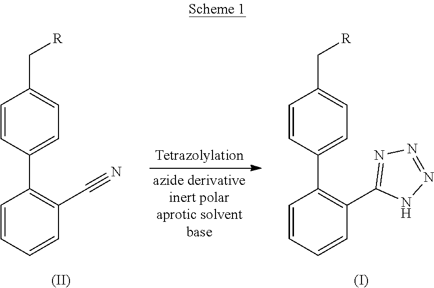

wherein R is as defined above.

Among the azide derivative may be particularly cited hydrazoic acid (HN₃), salt azide for instance a metal azide such as sodium azide (NaN₃), potassium azide (KN₃), or calcium azide (Ca (N₃)₂), SnBu₃N₃, SnMe₃N₃, trialkyl ammonium azide, such as triethylammonium azide, in particular a metal or salt azide, such as sodium azide or triethtylammonium azide.

Among the polar aprotic solvents having reductive properties may be cited N-methylformamide (MFo), N,N-dimethylformamide (DMF), N-methyl,N-Tert-butylformamide, acetamide (Ac), N-methylacetamide (MAc), N,N-dimethylacetamide (DMAc), urea, tetramethyl urea (TMU), dimethylpropylene urea (DMPU), dimethylethylene urea (DMEU), triethylamine (TEA), hexamethylphosphoramide (HMPA), hexamethylphosphorotriamide (HMPT), 2-pyrrolidone (2-Py), N-methyl-2-pyrrolidone (NMP), N-phenyl-2-pyrrolidone (NPP), N-vinylpyrrolidone (NVP), and 5-methyl-2-pyrrolidone (MPy).

Thus, herein is further provided a process according to the present disclosure, wherein said polar aprotic solvent having reductive properties is selected from N-methylformamide (MFo), N,N-dimethylformamide (DMF), N-methyl,N-Tert-butylformamide, acetamide (Ac), N-methylacetamide (MAc), N,N-dimethylacetamide (DMAc), urea, tetramethyl urea (TMU), dimethylpropylene urea (DMPU), dimethylethylene urea (DMEU), triethylamine (TEA), hexamethylphosphoramide (HMPA), hexamethylphosphorotriamide (HMPT), 2-pyrrolidone (2-Py), N-methyl-2-pyrrolidone (NMP), N-phenyl-2-pyrrolidone (NPP), N-vinylpyrrolidone (NVP), and 5-methyl-2-pyrrolidone (MPy).

The base may be selected from triethylamine (Et₃N), N,N-dicyclohexylmethylamine, and a Hunig base such as N,N-diisopropylethylamine and the like. Said base may in particular be triethylamine and more particularly triethylamine hydrochloride (also named TEA, HCl).

Use is preferably made of equimolecular amounts of alkali metal azide and of triethylamine hydrochloride in proportions of 1 to 5 moles per mole of starting nitrile, advantageously of about 1.2 to about 2 moles per mole nitrile.

During the tetrazolylation, the reaction medium can be heated at a temperature ranging between room temperature to 150° C., in particular 100° C. to 135° C., and for example 150° C.

After 6-20 hours of heating, the tetrazolylation is complete and the reaction mixture is worked up according to conventional techniques. In particular, the mixture is neutralized by adding a base, for example an alkali metal hydroxide, in aqueous solution, the aqueous phase containing the salts, in particular chlorides and azides, is discarded.

The organic phase is then treated with water and various organic solvents (aromatics, halogenated, esters, ketones, . . . ) such as toluene, ethyl acetate, dichloromethane (DCM), methylethylketone optionally with two different solvents sequentially, making it possible to remove the reaction by-products.

These washing steps are conventional and well known by the skilled person.

The final product is then crystallized via a crystallization step also well known by the skilled in the art. Additional conventional filtration and washing steps can then be performed if necessary.

Among the sartan active compounds of formula (I) as defined above may be cited irbesartan, losartan, valsartan, candesartan, or olmesartan, in particular irbesartan.

According to a particular embodiment, the sartan active compound of formula (I) as defined above is irbesartan, also called 2-n-butyl-4-spirocyclopentane-1-[[2′-(tetrazol-5-yl)biphenyl-4-yl]methyl]-2-imidazolin-5-one or 2-n-butyl-3-[[2′-(tetrazol-5-yl)biphenyl-4-yl]methyl]-1,3-diazaspiro[4.4]non-1-en-4-one.

Herein is further provided a process comprising at least the steps consisting of:
having a reactional mixture containing at least iron ions, in particular ferrous ions, and more particularly obtained in situ via ferric ions, an alkali metal azide and one base in a polar aprotic solvent having reductive properties below the reflux temperature and under inert atmosphere,
adding to said reactional mixture 2-n-butyl-1-[(2′-cyanobiphenyl-4-yl)methyl]-4-spirocyclopentan-2-imidazolin-5-one,
promoting said tetrazolylation, and
recovering the irbesartan thus obtained in the form of one of its alkali metal salts in aqueous solution.

Herein is further provided a process according to the present disclosure, wherein said polar aprotic solvent having reductive properties is selected from N-methylformamide (MFo), N,N-dimethylformamide (DMF), N-methyl,N-Tert-butylformamide, acetamide (Ac), N-methylacetamide (MAc), N,N-dimethylacetamide (DMAc), urea, tetramethyl urea (TMU), dimethylpropylene urea (DMPU), dimethylethylene urea (DMEU), triethylamine (TEA), hexamethylphosphoramide (HMPA), hexamethylphosphorotriamide (HMPT), 2-pyrrolidone (2-Py), N-methyl-2-pyrrolidone (NMP), N-phenyl-2-pyrrolidone (NPP), N-vinylpyrrolidone (NVP), and 5-methyl-2-pyrrolidone (MPy), in particular is 1-methylpyrrolidin-2-one, at a temperature of 80° C. to 150° C., in particular of 100° C. to 135° C.

When the sartan active compound of formula (I) is irbesartan, use may be particularly made of 1-methylpyrrolidin-2-one as a polar aprotic solvent having reductive properties, at a temperature of 80° C. to 150° C., in particular of 100° C. to 135° C.

Herein is further provided a process according to the present disclosure, wherein the so-obtained irbesartan contains less than 10 ppm, in particular less than 5 ppm of a compound of formula (B)

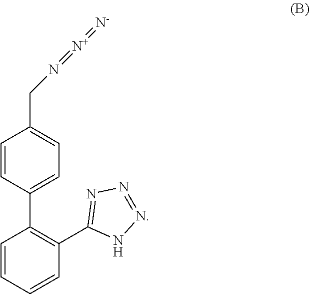 [SMILES: [N-]=[N+]=NCc1ccc(-c2ccccc2-c2nnn[nH]2)cc1]

Herein is further provided a process according to the present disclosure, wherein the so-obtained irbesartan contains less than 10 ppm, in particular less than 5 ppm and more particularly less than 1 ppm of a compound of formula (B1)

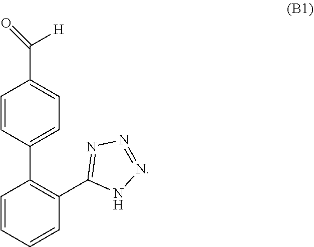 [SMILES: O=Cc1ccc(-c2ccccc2-c2nnn[nH]2)cc1]

The compound of formula (B1) may be then eliminated after performing conventional filtration and washings which are commonly carried out in synthesis involving tetrazolylation to obtain sartan compounds.

During the tetrazolylation, the nitrile group contained in a compound of formula (II) as defined in the present disclosure may be transformed into a tetrazole group to form irbesartan. The same reaction may be performed to convert compound of formula (A) into compound of formula (B). At the end of a conventional tetrazolylation, a compound of formula (A) is less than 1 ppm (detection limit) and only a compound of formula (B) remains.

The inventors have compared the amount of compound of formula (B1) obtained without the conversion of benzylic azide impurities into aldehydes derivatives and with the conversion of benzylic azide impurities into aldehydes derivatives for the synthesis of irbesartan.

It came out that with a manufacturing process not implementing the conversion of the benzylic azide impurities formed during said tetrazolylation according to the prior art, irbesartan obtained after the tetrazolylation step could present an amount of around 130 ppm (at the end of the tetrazolylation reaction) with respect to the total amount of irbesartan, while the manufacturing process implementing the conversion of the benzylic azide impurities formed during said tetrazolylation according to the present invention give an amount of less than 10 ppm, in particular less than 5 ppm, with respect to the total amount of irbesartan, prior to the final purification steps.

And even if afterwards conventional steps of purification were conducted, a manufacturing process according to the prior art, i.e. not implementing the conversion of the benzylic azide impurities formed during said tetrazolylation, irbesartan thus obtained could present an amount of 30±10 ppm with respect to the total amount of irbesartan, which remains well beyond the requirements of the health authorities.

A manufacturing process according to present invention, i.e. implementing an conversion of the benzylic azide impurities formed during said tetrazolylation, allows to provide an amount of less than 1 ppm of said benzylic azide impurities with respect to the total amount of irbesartan after the final purification steps.

### Further Impurities

In addition, the inventors have noted that further impurities different from aldehydic impurities could also be potentially formed, in particular because of the presence of the solvent in an oxidized form.

More particularly, when the sartan active compound (I) is irbesartan and that the inert aprotic polar solvent used was N-methyl-2-pyrrolidone, in addition to tetrazole aldehydic impurity, four NMP irbesartan adducts impurities were detected. Their presence is due to the reaction of oxidized NMP with irbesartan. These four adducts have the following formulas (C1), (D1), (E1) and (F1) as shown below:

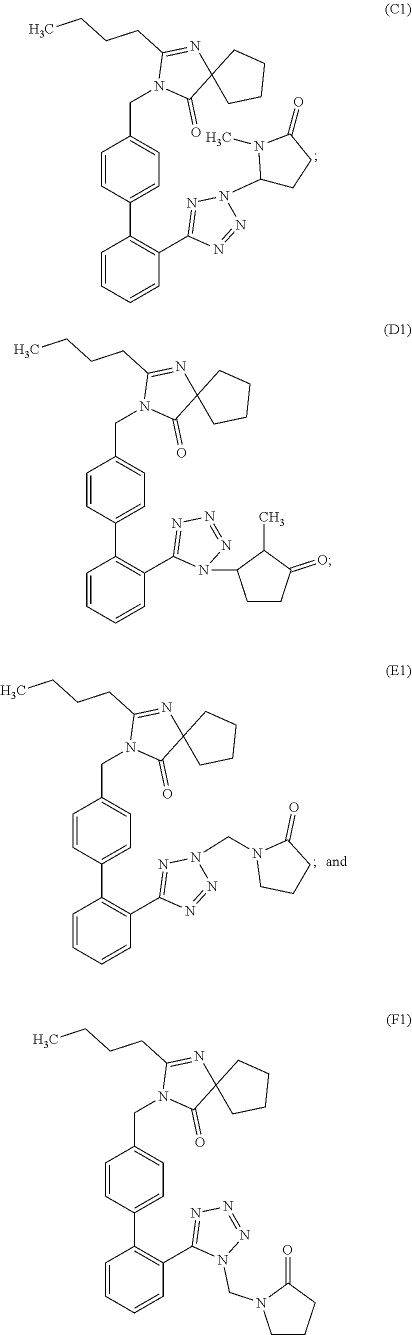 [SMILES: CCCCC1=NC2(CCCC2)C(=O)N1Cc1ccc(-c2ccccc2-c2nnn(C3CCC(=O)N3C)n2)cc1 ; CCCCC1=NC2(CCCC2)C(=O)N1Cc1ccc(-c2ccccc2-c2nnnn2C2CCC(=O)C2C)cc1 ; CCCCC1=NC2(CCCC2)C(=O)N1Cc1ccc(-c2ccccc2-c2nnn(CN3CCCC3=O)n2)cc1 ; CCCCC1=NC2(CCCC2)C(=O)N1Cc1ccc(-c2ccccc2-c2nnnn2CN2CCCC2=O)cc1]

However, as for the tetrazole aldehydic impurity of formula (B1) as defined above, these four irbesartan adducts were assessed as a class 5 impurity, which means that they were considered as non-mutagenic compounds. Their joint presence in the final product is thus no detrimental in view of the requirements of the health authorities.

Hereinafter, the present invention will be described in more details, with reference to the following examples. These examples are provided to illustrate the present invention and should not be construed as limiting the scope and spirit of the present invention.

### EXAMPLES

The ¹H NMR spectra are recorded on a Bruker AC200 spectrometer operating at 200 MHz, the ¹HRVN spectrum of a 2 percent (m/V) solution of irbesartan in hexadeuterated dimethyl sulphoxide (DMSO-d6) containing tetramethylsilane, as the internal standard, referenced at 2.5 ppm at a temperature of 300 K. The chemical shifts are calibrated with respect to the TMS signal (the signal at 0 ppm is due to the internal standard tetramethylsilane).

Or, the ¹H NMR spectra are recorded on a Bruker Avance III spectrometer operating at 400 MHz, with the chemical shifts (S in ppm) using the solvent CDCl3 referenced at 7.26 ppm at a temperature of 300 K. The chemical shifts are calibrated with respect to the TMS signal (the signal at 0 ppm is due to the internal standard tetramethylsilane).

### Example 1: Preparation of Crude Irbesartan by a Process According to the Disclosure and Quantification of Impurities (Compounds of Formulas (A), (B) and (B1))

### Example 1-1: Preparation of Crude Irbesartan

In a suitable reactor which is inerted by nitrogen gas, a mixture of 35.2 g of triethylamine hydrochloride, 1.5 mg of FeCl₃, 82 g of N-methylpyrrolidone, 16.8 g of sodium azide and 80 g of 2-N-butyl-1-[(2′-cyanobiphenyl-4-yl)methyl]-4-spirocyclopentane-2-imidazoline-5-one was gradually heated to a temperature of approximately 130-135° C.

Then, the mixture was maintained at a temperature of approximately 130-135° C. for at least 14 hours.

The mixture was then cooled to 20-30° C., and 12 g N-methylpyrrolidone, 127 g of toluene and 11 g of water were added. Then the mixture was washed with a 30 w/w % aqueous sodium hydroxide solution repeatedly.

Water was then added and the reaction medium was acidified with concentrated hydrochloric acid to a pH of approximately 4 at 10 to 15° C. The organic phase was then discarded and the remaining solid material washed with water.

The organic layer was then poured on a 8 w/w % aqueous sodium hydroxide solution at about 5 to 10° C. After stirring 15 min, organic phase was discarded and the aqueous layer was washed with about 80 ml of toluene.

122 ml of Methyl ethyl ketone and 12 ml of water were added and pH was adjusted to 8 with concentrated HCl at about 15° C., filtrated through a charcoal cartridge, heated to 50° C. and pH was adjusted to 7 with HCl.

After seeding and acidification to pH 4, the slurry was cooled to 25° C., maintained 1 h and filtrated.

The cake was washed with iso-Propanol repeatedly and dried to deliver the pure irbesartan.

The ¹H RMN characterization for irbesartan is as follows:

¹H RMN (200 MHz, δ in ppm, DMSO): 0.8 (T, 3H); 1.3 (sextuplet, J=8.0 Hz, 2H); 1.5 (q, 2H); 1.6 to 2.0 (m, 8H); 2.3 (t, 2H); 4.7 (s, 2H); 7.1 (s 4H); 7.4 to 7.8 (m, 4H)

### Example 1-2: Quantification of Compounds of Formulas (A), (B) and (B1)

The ¹H RMN characterization for compounds of formulas (A), (B) and (B1) are as follows:

For compound of formula (A):

¹H RMN (400 MHz, δ in ppm, CDCl₃): 4.34 (s, 2H); 7.12 (d, J=8.0 Hz, 2H); 7.25 (d, J=8.0 Hz, 2H); 7.43 (d, J=7.5 Hz, 1H); 7.50 (t, J=7.5 Hz, 1H); 7.60 (t, J=7.5 Hz, 1H); 7.91 (d, J=7.5 Hz, 1H)

For compound of formula (B):

¹H RMN (400 MHz, δ in ppm, CDCl₃): 4.42 (s, 2H); 7.44 (d, J=8.0 Hz, 2H); 7.46 (m, 1H); 7.51 (d, J=8.0 Hz, 1H); 7.58 (d, J=8.0 Hz, 2H); 7.65 (td, J=7.5 Hz, J=1.5 Hz, 1H); 7.77 (d, J=7.5 Hz, 1H)

For compound of formula (B1):

¹H RMN (400 MHz, δ in ppm, DMSO): 7.32 (d, J=8.0 Hz, 2H); 7.55 (d, J=7.5 Hz, 1H); 7.59 (t, J=7.5 Hz, 1H); 7.66 (t, J=7.5 Hz, 1H); 7.73 (d, J=7.5 Hz, 2H); 7.83 (d, J=8.0 Hz, 2H); 10.00 (s, 1H)

The quantity of impurities of formulas (A), (B) and (B1) were measured at two moments:
firstly, at the end of the tetrazolylation reaction as performed in example 1-1 above, and
secondly, when the pure isolated irbesartan was obtained according to example 1-1 above.

The analytical method used for measuring the quantity of compounds of formula (A) and (B) was as follows:

The analysis was performed by UPLC.

### For the Liquid Chromatography Part (UPLC Vanquish Thermo Scientific):

The chromatography was performed by using the column with the following characteristics

| Items | characteristics | |
|---|---|---|
| Phase | Acquity HSS T3 | |
| Length | 100 | mm |
| Diameter | 2.1 | mm |
| Granulometry | 1.8 | μm |

| | |
|---|---|
| Provider | Waters-reference: 186003539 |

a temperature of column: 50° C.
a temperature of the sample changer: 25° C.
an injected volume: 2 μL
a spectrophotometric detector set to 254 nm (optional)
a mobile phase containing a mixture:

A: H₂O+0.1% formic acid (v/v) and

B: methanol,

the gradient of which being as follows:

| Time (min) | A (%) | B (%) | Debit (mL/min) |
|---|---|---|---|
| 0 | 50 | 50 | 0.5 |
| 5 | 50 | 50 | 0.5 |
| 10 | 40 | 60 | 0.5 |
| 11 | 40 | 60 | 0.5 |
| 11.1 | 5 | 95 | 0.5 |
| 13 | 5 | 95 | 0.5 |
| 13.1 | 50 | 50 | 0.5 |
| 16 | 50 | 50 | 0.5 |

The chromatography was performed by using the following switching valve:

| Time of analysis | Position of the valve |
|---|---|
| 0 | 1-2 towards MS (mass spectrometry) |

| | | |
|---|---|---|
| 5 | min | 1-6 towards dustbin |
| 7.25 | min | 1-2 towards MS (mass spectrometry) |

The time of the analysis was 16 min.

### For the Mass Spectrometry Part (Q Exactive Thermo Fischer Scientific):

The quantification was carried out by using:
an ionization mode: HESI
a voltage spray: 3 500 V
a capillary temperature: 300° C.
a sheat gas: 70 arbitrary unit
an auxiliary gas: 20 arbitrary unit
a sweep gas: 0 arbitrary unit
a temperature of auxiliary gas: 300° C.
S-lens RF level: 50 arbitrary unit

### The Mode of Detection for Compounds of Formulas (A) and (B) is as Follows:

PRM (Parallel Reaction Monitoring) in positive mode with extraction of fragments m/z (resolution 17 500) with the inclusion list:

| | Compound of | Compound of |
|---|---|---|
| | formula (A) | formula (B) |
| Mass (m/z) | 207.09167 | 278.11487 |
| Formula | C14H10N4 | C14H11N7 |
| Mode | +H | +H |
| Polarity | Positive | Positive |
| Start (min) | 7.25 | 0 |
| End (min) | 16 | 5 |
| Collision energy | 35 | 35 |
| Resolution | 17 500 | 17 500 |
| Isolation window | 2 | 2 |
| for the quadrupole | | |

Sought Ions for Compounds of Formulas (A) and (B):

| Compounds | Filter | Mass (m/z) |
|---|---|---|
| Formula (A) | FTMS + p ESI Full | 207.09167 |
| | ms2 207.09@hcd35.00 [50.00-230.00] | |
| Formula (B) | FTMS + p ESI Full | 207.09167 |
| | ms2 278.11@hcd35.00 [50.00-300.00] | |

The analytical method used for measuring the quantity of compounds of formula (B1) was as follows:

The analysis was performed in LC-MS with LC-MS SRDA-UC09-FUSION (column acquity HSS C18-50*2.1 mm-1.8 μm, Mobile phase A=H₂O and Mobile phase B=methanol, debit=0.8 mL/min, column temperature=50° C., Gradient=from 5% B to 90% B in five minutes, mass detection: positive ESI-SIM on m/z=251 and m/z=267.

The results are gathered in the following table 1.

TABLE 1

| | End the of | Pure isolated |
|---|---|---|
| | tetrazolylation | Irbesartan |
| | reaction | obtained |
| Quantity of compound | <1 ppm versus | <1 ppm versus |
| of formula (A) | irbesartan | irbesartan |
| Quantity of compound | 5 ppm versus | 1 ppm versus |
| of formula (B) | irbesartan | irbesartan |
| Quantity of compound | 50 ppm versus | <5 ppm versus |
| of formula (B1) | irbesartan | irbesartan |

It comes out from these results that a process in accordance with the present disclosure allows to degrade azido impurities, more particularly allows to convert the compound of formula (B) into the compound of formula (B1).

In addition, it was shown that filtration and purification steps carried out after the tetrazolylation in order to obtain the pure isolated irbesartan allow to reduce both the quantity of the compound of formula (B) (from 5 ppm to 1 ppm) and the compound of formula (B1) (from 50 ppm to less than 5 ppm).

### Example 2 (Comparative): Preparation of Crude Irbesartan by a Process Outside the Disclosure (that is to Say without the Presence of Ferrous Ions in the Medium) and Quantification of Impurities (Compounds of Formulas (A) and (B))

### Example 2-1: Preparation of Crude Irbesartan by a Process Outside the Disclosure (without FeCl₃)

A process analogous to the process as described in example 1 above was performed, except that no FeCl₃ was used. The details are explained below.

In a suitable reactor which is inerted by nitrogen gas, a mixture of 35.2 g of triethylamine hydrochloride, 82 g of N-methylpyrrolidone, 16.8 g of sodium azide and 80 g of 2-N-butyl-1-[(2′-cyanobiphenyl-4-yl)methyl]-4-spirocyclopentane-2-imidazoline-5-one was gradually heated to a temperature of approximately 130-135° C.

Then, the mixture was maintained at a temperature of approximately 130-135° C. for at least 14 hours.

The mixture was then cooled to 20-30° C., and 12 g N-methylpyrrolidone, 127 g of toluene and 11 g of water were added. Then the mixture was washed with a 30 w/w % aqueous sodium hydroxide solution repeatedly.

Water was then added and the reaction medium was acidified with concentrated hydrochloric acid to a pH of approximately 4 at 10 to 15° C. The organic phase was then discarded and the remaining solid material washed with water.

The organic layer was then poured on a 8 w/w % aqueous sodium hydroxide solution at about 5 to 10° C. After stirring 15 min, organic phase was discarded and the aqueous layer was washed with about 80 ml of toluene.

122 ml of Methyl ethyl ketone and 12 ml of water were added and pH was adjusted to 8 with concentrated HCl at about 15° C., filtrated through a charcoal cartridge, heated to 50° C. and pH was adjusted to 7 with HCl.

After seeding and acidification to pH 4, the slurry was cooled to 25° C., maintained 1 h and filtrated.

The cake was washed with iso-Propanol repeatedly and dried to deliver the pure irbesartan.

The ¹H RMN characterization for irbesartan is as follows:

¹H RMN (200 MHz, δ in ppm, DMSO): 0.8 (T, 3H); 1.3 (sextuplet, J=8.0 Hz, 2H); 1.5 (q, 2H); 1.6 to 2.0 (m, 8H); 2.3 (t, 2H); 4.7 (s, 2H); 7.1 (s 4H); 7.4 to 7.8 (m, 4H)

### Example 2-2: Quantification of Compounds of Formulas (A) and (B)

The ¹H RMN characterization for compounds of formulas (A), and (B) are as follows:

For compound of formula (A):

¹H RMN (400 MHz, δ in ppm, CDCl₃): 4.34 (s, 2H); 7.12 (d, J=8.0 Hz, 2H); 7.25 (d, J=8.0 Hz, 2H); 7.43 (d, J=7.5 Hz, 1H); 7.50 (t, J=7.5 Hz, 1H); 7.60 (t, J=7.5 Hz, 1H); 7.91 (d, J=7.5 Hz, 1H)

For compound of formula (B):

¹H RMN (400 MHz, δ in ppm, CDCl₃): 4.42 (s, 2H); 7.44 (d, J=8.0 Hz, 2H); 7.46 (m, 1H); 7.51 (d, J=8.0 Hz, 1H); 7.58 (d, J=8.0 Hz, 2H); 7.65 (td, J=7.5 Hz, J=1.5 Hz, 1H); 7.77 (d, J=7.5 Hz, 1H)

The quantity of impurities of formulas (A) and (B) were measured at two moments:
firstly at the end of the tetrazolylation reaction as performed in example 2-1 above, and
secondly when the pure isolated irbesartan was obtained according to example 2-1 above.
   The analytical method used for measuring the quantity of compounds of formulas (A) and (B) was similar to that used and described in example 1-2 above.

The results are gathered in the following table 2.

TABLE 2

| | End the of | Pure isolated |
|---|---|---|
| | tetrazolylation | Irbesartan |
| | reaction | obtained |
| Quantity of compound | <1 ppm versus | <1 ppm versus |
| of formula (A) | irbesartan | irbesartan |
| Quantity of compound | 130 ± 20 ppm | 30 ± 10 ppm |
| of formula (B) | versus irbesartan | versus irbesartan |

By comparing the results of example 1-2 and 2-2, it comes out that:
the presence of ferrous ions in a process in accordance with the disclosure allows to decrease significantly the quantity of the compound of formula (B) both at the end of the tetrazolylation reaction (5 ppm versus 130±20 ppm) and when pure isolated irbesartan is obtained (1 ppm versus 30±10 ppm).
The weak quantity of the compound of formula (A) is similar in both processes,
The compound of formula (B1) is only obtained by carrying out a process in accordance with the present disclosure, showing that the compound of formula (B) is converted into the compound of formula (B1) thanks to the presence of ferrous ions in the medium.

### Example 3: Degradation of a Compound of Formula (B) into a Compound of Formula (B1) in the Presence of FeCl₃

### Example 3-1

To 0.11 g of a compound of formula (B) were added 4.11 g of NMP and 0.013 g of FeCl₃ which were heated at 135° C. during 2 hours by stirring with a magnetic stirrer.

The detection of the presence of the compounds of formulas (B) and (B1) was carried out by LC-MS with LC-MS SRDA-UC09-FUSION equipment with a column XBridge C18 (100*4.6 mm-3.5 μm) and a gradient ammonium acetate 10 mM pH4.5/acetonitrile.

At 6.6 min, a majority peak was observed (UV 250 nm) which corresponded to a compound of formula (B1) and at 8.33 min, the compound of formula (B) was not detected anymore.

### Example 3-2

Two solutions were prepared as follows:
1) Solution 1: 11.1 mg of compound (B) was added in 6 ml of NMP and then solubilized (1850 ppm)
2) Solution 2: 8.8 mg of FeCl₃ was added in 1 mL of NMP and then solubilized

A blank and two samples were then prepared and loaded in three different vials as follows:
Blank: 2 mL of solution 1 (vial 1)
Sample 1: 2 mL of solution 1 with 50 μL of solution 2 (that is to say 0.2 eq) (vial 2)
Sample 2: 2 mL of solution 1 with 50 μL of solution 2 (that is to say 0.2 eq), and argon was bubbled before closing the vial and heating (vial 3).

The three reaction mediums were colored after 15 min of heating and were heated at 135° C. during 3 hours.

The results are gathered in table 3 as follows:

TABLE 3

| Sample/vial | Compound (B) content (ppm) |
|---|---|
| Blank - vial 1 | 119 |
| Sample 1 - Vial 2 | <1 |
| Sample 2 - Vial 3 | <1 |

It comes out from the results that the profiles are the same for sample 1 and sample 2, which means that the presence or absence of oxygen has no impact and that compound (B) disappears in the presence of FeCl₃.

The example 3 thus proves the degradation of azido tetrazole impurity of formula (B) into tetrazole aldehydic impurity of formula (B1) which occurs due to the presence of FeCl₃.

This example also allows to prove that the conversion of a compound of formula (B) into a compound of formula (B1) can be carried out either simultaneously or subsequently to the tetrazolylation reaction.

### Example 4: Degradation of the Compound of Formula (B) in the Presence of FeCl₂

In a suitable vessel, 1.47 g of compound of formula (B), 34 mg of FeCl₂ and 800 ml of N-methylpirrolidone were heated to 100° C. for 3 hours.

Reaction mixture was cooled to room temperature and analyzed via HPLC, offering a degradation of the compound of formula (B), by observation of the disappearance of the corresponding peak.

The results are as follows:
Compound (B) before the reaction: 100 area % at RT 3.2 min
Compound (B) after 3 hours at 100° C.: 5.1 area % at RT 3.2 min beside a major degradant at 1.3 min representing 54 area %

The operating conditions were as follows.

### Column:

Phase: X-Bridge Cis

Length: 100 mm

Diameter: 4.6 mm

Granulometry: 3.5 μm

Provider: Waters ref. 186003033

Temperature of the column: 35° C.
Temperature of the injector: room temperature
Mobile phase:
A=aqueous solution of ammonium acetate 10 mM (for example 770 mg in 1 L of H₂O) adjusted at pH=4,5 with a diluted aqueous solution of acetic acid.
B=Acetonitrile.

### Gradient:

| Time | A | B |
|---|---|---|
| T = 0 min | 67% | 33% |
| T = 1.6 min | 67% | 33% |
| T = 6.9 min | 41% | 59% |
| T = 12.0 min | 41% | 59% |
| T = 13.0 min | 67% | 33% |
| T = 18.0 min | 67% | 33% |

Debit: 1.2 ml/min
Injected volume: 5 μl

### Detection: 250 nm

Duration of the analysis: 12 min
Equilibrium time: 5 min

1. A process for manufacturing at least one sartan active compound of formula (I) 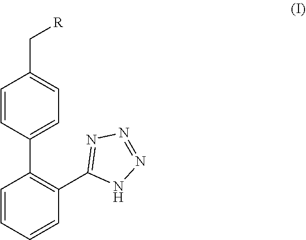
wherein R is selected from a group of formulas (1), (2), (3), (4) and (5): 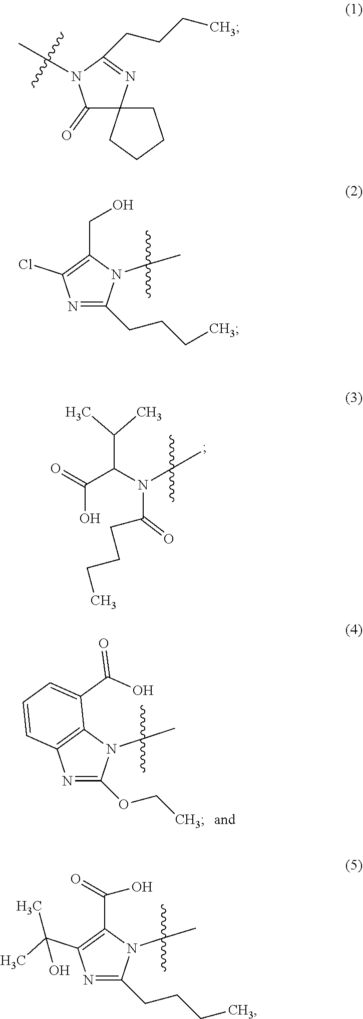 [symbol]
being the attachment site,
comprising the tetrazolylation of one compound of formula (II) 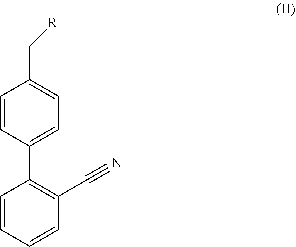
wherein R is as previously defined,
in a reactional medium with at least one azide derivative, wherein benzylic azide impurities formed during said tetrazolylation are converted into aldehyde derivatives.

2. The process according to claim 1, wherein said benzylic azide impurities are converted into aldehyde derivatives by performing an oxidation step followed by a hydrolysis step.

3. The process according to claim 1, wherein said benzylic azide impurities are converted into aldehyde derivatives by contacting said benzylic azides impurities with at least ferrous ions.

4. The process according to claim 4, wherein said ferrous ions are formed in situ by reduction of ferric ions, in particular in the presence of a polar aprotic solvent having reductive properties.

5. The process according to claim 4, wherein said ferric ions are generated from FeCl₃, FePO₄, FeI₃, FeF₃, FeBr₃, Fe₂(SO₄)₃, Fe₂(C₂O₄)₃, Fe(OH)₃, FeCl₃.6H₂O, FeF₃.3H₂O, Fe₄(P₂O₇)₃, Fe₄(Fe(CN)₆)₃, or Fe(H₂PO₂)₃, in particular from FeCl₃, FePO₄, FeI₃, FeF₃, FeBr₃, Fe₂(SO₄)₃, Fe₂(C₂O₄)₃, Fe(OH)₃, for instance FeCl₃.

6. The process according to claim 4, wherein said polar aprotic solvent having reductive properties is selected from N-methylformamide (MFo), N,N-dimethylformamide (DMF), N-methyl,N-Tert-butylformamide, acetamide (Ac), N-methylacetamide (MAc), N,N-dimethylacetamide (DMAc), urea, tetramethyl urea (TMU), dimethylpropylene urea (DMPU), dimethylethylene urea (DMEU), triethylamine (TEA), hexamethylphosphoramide (HMPA), hexamethylphosphorotriamide (HMPT), 2-pyrrolidone (2-Py), N-methyl-2-pyrrolidone (NMP), N-phenyl-2-pyrrolidone (NPP), N-vinylpyrrolidone (NVP), and 5-methyl-2-pyrrolidone (MPy).

7. The process according to claim 1, wherein said benzylic azide impurities are converted into aldehyde derivatives simultaneously to said tetrazolylation.

8. The process according to claim 3, wherein said ferrous ions are present at a catalytic amount in the basic medium containing the azide derivative, in particular in a molar percentage ranging from 0.005% to 0.1%, in particular from 0.01% to 0.05% with respect to the amount of the compound of formula (II).

9. The process according to claim 1, wherein said benzylic azide impurities are converted into aldehyde derivatives subsequently to the tetrazolylation.

10. The process according to claim 1, wherein said benzylic azide impurities comprise at least the two following compounds of formulas (A) and (B)

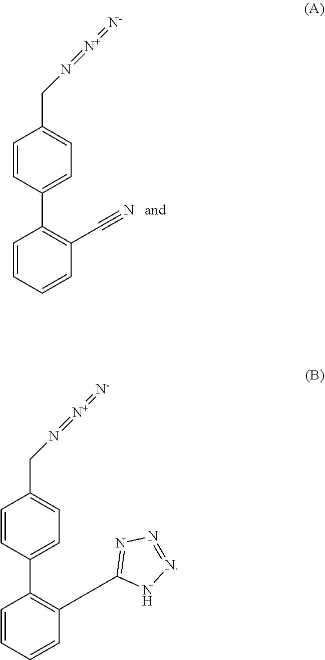 [SMILES: N#Cc1ccccc1-c1ccc(CN=[N+]=[N-])cc1 ; [N-]=[N+]=NCc1ccc(-c2ccccc2Cc2nnn[nH]2)cc1]

11. The process according to claim 1, wherein the so-obtained aldehyde derivatives comprise at least the compound of formula (B1)

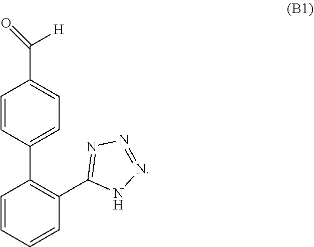 [SMILES: O=Cc1ccc(-c2ccccc2-c2nnn[nH]2)cc1]

12. The process according to claim 1, wherein the obtained sartan active compound of formula (I) contains less than 10 ppm, in particular less than 5 ppm, and more particularly less than 1 ppm of a compound of formula (B1)

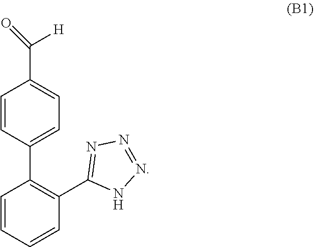 [SMILES: O=Cc1ccc(-c2ccccc2-c2nnn[nH]2)cc1]

13. The process according to claim 1, wherein the sartan active compound of formula (I) is irbesartan, also called 2-n-butyl-4-spirocyclopentane-1-[[2′-(tetrazol-5-yl)biphenyl-4-yl]methyl]-2-imidazolin-5-one or 2-n-butyl-3-[[2′-(tetrazol-5-yl)biphenyl-4-yl]methyl]-1,3-diazaspiro[4.4]non-1-en-4-one.

14. The process according to claim 13, comprising at least the steps of:
having a reactional mixture containing at least iron ions, in particular ferrous ions, and more particularly obtained in situ via ferric ions, an alkali metal azide and one base in a polar aprotic solvent having reductive properties below the reflux temperature and under inert atmosphere,
adding to said reactional mixture 2-n-butyl-1-[(2′-cyanobiphenyl-4-yl)methyl]-4-spirocyclopentan-2-imidazolin-5-one,
promoting said tetrazolylation, and
recovering the irbesartan thus obtained in the form of one of its alkali metal salts in aqueous solution.

15. The process according to claim 14, wherein said polar aprotic solvent having reductive properties is selected from N-methylformamide (MFo), N,N-dimethylformamide (DMF), N-methyl,N-Tert-butylformamide, acetamide (Ac), N-methylacetamide (MAc), N,N-dimethylacetamide (DMAc), urea, tetramethyl urea (TMU), dimethylpropylene urea (DMPU), dimethylethylene urea (DMEU), triethylamine (TEA), hexamethylphosphoramide (HMPA), hexamethylphosphorotriamide (HMPT), 2-pyrrolidone (2-Py), N-methyl-2-pyrrolidone (NMP), N-phenyl-2-pyrrolidone (NPP), N-vinylpyrrolidone (NVP), and 5-methyl-2-pyrrolidone (MPy), in particular is 1-methylpyrrolidin-2-one at a temperature of 80° C. to 150° C., in particular of 100° C. to 135° C.

16. The process of claim 14, wherein the so-obtained irbesartan contains less than 10 ppm, in particular less than 5 ppm of a compound of formula (B)

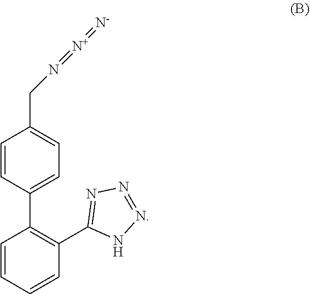 [SMILES: [N-]=[N+]=NCc1ccc(-c2ccccc2-c2nnn[nH]2)cc1]

17. The process of claim 14, wherein the so-obtained irbesartan contains less than 10 ppm, in particular less than 5 ppm and more particularly less than 1 ppm of a compound of formula (B1) 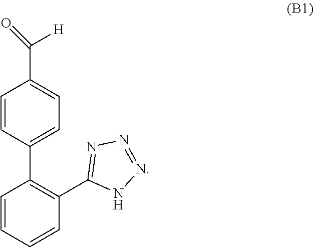 [SMILES: O=Cc1ccc(-c2ccccc2-c2nnn[nH]2)cc1]
